(19) United States
(12) Patent Application Publication
(10) Pub. No.: US 2013/0018238 A1
(43) Pub. Date: Jan. 17, 2013

(54) Enhanced non-invasive analysis system and method

(71) Applicants: Andrew Patti, Cupertino, CA (US); Carol Jean Wilson, San Jose, CA (US); Josh N. Hogan, Los Altos, CA (US)

(72) Inventors: Andrew Patti, Cupertino, CA (US); Carol Jean Wilson, San Jose, CA (US); Josh N. Hogan, Los Altos, CA (US)

(21) Appl. No.: 13/199,932

(22) Filed: Sep. 13, 2011

Related U.S. Application Data
(60) Provisional application No. 61/403,327, filed on Sep. 14, 2010.

(51) Int. Cl. A61B 6/02; A61B 5/1455

(52) U.S. Cl. 600/316; 600/425; 600/310

(57) ABSTRACT

The invention provides an enhanced method and system for non-invasive analysis of a target. The enhancement includes increased analytic power derived from creating a complete representation of a target using less than complete information. The invention provides a non-invasive analysis system and method that includes generating and exploiting a system model that includes a target model that accurately represents the interaction of radiant energy with a target.

In a preferred embodiment according to the invention, a digital signal processor compares signals acquired from an actual non-invasive system with theoretical signals generated using the system model, identifies the target model that matches most closely, and outputs target characteristics, including target attribute of interest.

### RELATED APPLICATIONS

This patent application, docket number FP100801, claims priority from U.S. provisional application 61/403,327 of the same title and by the same inventors, file date Sep. 14, 2010, the entirety of which is incorporated by reference as if fully set forth herein. This patent application is also related to U.S. application Ser. No. 11/818,309, (Publication number US2007/0260128), the entirety of which is incorporated by reference as if fully set forth herein. This application further relates to U.S. patent application Ser. No. 12/584,666 and PCT/US09/005,088 (“Noise Tolerant Measurement”) and to U.S. Pat. No. 7,248,907, European patent application EPO 05819669-2 and JPO 2007-538123 (“Correlation of Concurrent Nen-Invasively Acquired Signals”), the entireties of which are incorporated by reference as if fully set forth herein.

### GOVERNMENT FUNDING

None

### FIELD OF USE

The invention relates to application of interferometric techniques, such as OCT, for non-invasive analysis of a target. More particularly the invention relates to generating from partial interferometric target data a more complete representation of the target.

### BACKGROUND

Non-invasive analysis of a target is preferable to invasive analysis in many applications. Some powerful non-invasive techniques are under utilized, as the data obtained falls short of interferometrically obtaining complete target information.

The multiple depth scanning technique described in U.S. Pat. No. 7,526,329 and patent application Ser. No. 11/048,694 (incorporated herein by reference) yields incomplete target information in regions not covered by the one of the multiple references. Furthermore, lateral scanning is typically either a stepped scan or an effectively stepped raster scan which again yields incomplete information. Existing OCT systems also typically yield incomplete information regarding the target by the nature of the scanning or detection method. For example while typical time domain OCT systems can perform continuous depth scans their lateral scanning is also typically either a stepped scan or an effectively stepped raster scan.

Fourier domain OCT systems also typically have a stepped lateral scan. In the case of Fourier domain OCT systems that employ a detector array to simultaneously detect separated wavelengths, the segmented nature of the detector array yields incomplete information regarding the target. Each of these techniques provide incomplete information about a target. Thus, there is therefore an unmet need for a solution that can generate a more accurate representation of a target from incomplete information.

In non-interferometric techniques for in vivo tissue analysis, current approaches to characterizing tissue using interferometric techniques encounter difficulties in precisely identifying tissue components. While correlation of concurrently acquired signals exists, signal processing of interferometeric data is complex, and the amount of data needed as well as the amount of processing time impede the usefulness of tissue readings as a diagnostic or other analysis. One example would be determining glucose concentration in tissue non-invasively. Another application in the ophthalmic related tissue readings, such deformation of a retina, a lens or other eye component. Yet another example would be determining whether and the extent to which skin elements are likely to be malignant.

A widely appreciated example is non-invasive glucose monitoring. Glucose concentration in humans and other entities can be measured non-invasively using optical coherence tomography (OCT). OCT typically uses a super-luminescent diode (SLD) as the optical source, as described in Proceedings of SPIE, Vol. 4263, pages 83-90 (2001). The SLD output beam has a broad bandwidth and short coherence length. Another example easy to appreciate is the ophthalmic application where image or opto-metric information can be useful.

The OCT technique involves splitting the output beam into a probe and reference beam. The probe beam is applied to the system to be analyzed (the target). Light scattered back from the target is combined with the reference beam to form the measurement signal. Because of the short coherence length only light that is scattered from a depth within the target such that the total optical path lengths of the probe and reference are equal combine interferometrically. Thus the interferometric signal provides a measurement of the scattering value at a particular depth within the target. By varying the length of the reference path length, a measurement of the scattering values at various depths can be measured and thus the scattering value as a function of depth can be measured.

An alternative approach which generates interference signals from multiple depths simultaneously or concurrently is described in U.S. Pat. No. 7,526,329 and patent application Ser. No. 11/048,694 incorporated herein by reference. Scattering profile information can be generated by processing these interference signals. The correlation between blood glucose concentration and optical scattering by tissue has been reported in Optics Letters, Vol. 19, No. 24, Dec. 15, 1994 pages 2062-2064. The change of the scattering coefficient correlates with the glucose concentration and therefore measuring the change of the scattering value with depth (or scattering profile) provides a measurement of the scattering coefficient which provides a measurement of the glucose concentration. However this approach is negatively affected by having incomplete information due to the segmented nature of the scan.

A further unmet need is for a system capable of creating and using a model or representation of a target, including human tissue, as well as representation of noise sources, so that actual signals may be compared with theoretical or stored signal data and a more accurate representation of the target generated.

A further unmet need is a means to generate a representation of tissue that simulates actual structures within tissue so that signal analysis is simplified owing to reduction of the number of parameters that represent the tissue. A further unmet need is a means to use simulated combinations of individual scatterers and aggregates of scatterers to identify actual tissue structures such as cells or membranes, etc. to enable realistic multi-dimensional representation of actual tissue.

A further unmet need is an approach suitable to various radiations, such as ultrasound. Yet a further unmet need is a method of tissue analysis that, for example, is useful for analytes of interest, such as, for example, glucose, so as to develop and store realistic maps of tissue regions and use the map data to more accurately measure changes and provide analyte measurements.

What is further desirable is a means to output interferometrically acquired data to aid in characterization, diagnostics, detection, treatment, monitoring or otherwise related to tissue or other target analysis.

A further unmet need is a non-invasive means to monitor changes over time in structure of tissue features (optical biopsy), useful in applications such as, for example, relating to skin cancers and other skin conditions.

### BRIEF SUMMARY OF INVENTION

The invention taught herein meets at least all of the aforementioned unmet needs. The invention provides a system and method whereby partial target profile information is used to generate a more complete target profile or representation of the target. The invention provides a non-invasive interferometric analysis system and method that includes a system model that accurately represents the interaction of radiation with the target of interest.

In a system according to the preferred embodiment, the invention provides a non-invasive analysis system which is comprised of: an actual analysis system, a system model, a processor and an output means.

In an alternate embodiment, where the system model includes a multi-dimensional target model, the inventive system includes a display means, operable to image target information or depiction, where the User may select any portion or orientation of the target to display.

In the preferred embodiment the actual signals are interferometric signals, or preprocessed versions of interferometric signals, that are output from an actual measurement system, in particular interferometric signals created by an OCT measurement system. The interferometric signals are detected as analog signals and typically digitized and undergo pre-processing where such pre-processing may include, but is not limited to, filtering, Fourier analysis, envelope detection and the like, and are output to a Processor.

In the preferred embodiment, the system model is a parametric model providing actual system characteristics, as well as a target model.

In the embodiment where the non-invasive analysis system is an OCT, the system model includes the characteristics of the OCT system, such as center wavelength, bandwidth, power, focusing and scan rate and magnitude aspects, or equations that represent the OCT system.

The system model includes a representation of the target, where the representation includes a model of radiation interacting with the target. Interactions of radiation with the target include any or all of scattering, reflection and absorption and transmissive characteristics of the target.

In another embodiment, the system model includes representations of various noise sources, such as, optical source noise, mechanical noise, motion noise, detector noise, electronic noise, etc.

The system model generates and outputs to a processor at least one theoretical signal representing the interaction of radiation with the target. The theoretical signals generated by the system model are an ideal representation of the signals resulting from the interaction of radiation with an ideal target. For the purposes of this invention, an ideal target is a model that simulates actual structures within tissue.

The processor, which may be a microprocessor or DSP (digital signal processor), such as an ARM or one of the Blackfin processor family manufactured by Analog Devices, receives the actual signals and the theoretical signals.

In a preferred embodiment of the invention, the processor compares the theoretical signal with the actual signal, and determines the target model providing the best match to the actual signal.

In an alternate embodiment, the processor iteratively adjusts the parameters of the system model so that the difference between the actual and theoretical signals is minimized, and outputs information about at least one attribute, feature, or statistical distribution data relating to the target.

In an alternate embodiment of the invention, the target model includes grouped target components with known interactions with radiation. The processor can use such grouped characteristics to generate a target profile or representation better approximating a complete profile or representation from signals obtained from only a portion of the target.

The inventive system and method determine some attribute of the target. In embodiments discussed herein, attributes of interest generally belonging to three types: a) analytes of interest (an illustrative example could be glucose concentration in tissue),

b) images generated from the target profile generated from the system model, so that with incomplete interferometric data, a more complete representation of the target generated from the target profile enables a display of some User selected portion or orientation of the target (an illustrative example could be tomographic or 3D projection of ophthalmic tissue elements or structure, such as the inter-ocular lens, cornea or retina, etc.), and
c) statistical characteristics (an illustrative example would be cell or other structure size distribution).

The inventive method and system may be used to enhance a variety of non-invasive analysis instruments and techniques. In addition to OCT, a variety of spectral analysis and measurement devices may perform and benefit from the invention taught herein. Any instrument performing a non-continuous or segmented scan can practice the invention. Examples of non-continuous scans include lateral stepped scans. Additionally, Fourier domain OCT systems may use stepped tunable optical sources, such as stepped tunable laser diode or may be effectively segmented through use of segmented linear array detector. All of these are included for the purposes of this invention as “segmented scans”.

### BRIEF DESCRIPTION OF THE DRAWINGS

The drawings included herewith are:

FIG. 1 depicts an actual analysis system, such as OCT system analyzing tissue and generating actual signals that contain glucose-related information.

FIG. 2 is an illustration of a non-invasive analysis system according to the invention, which is comprised of an actual analysis system (as in FIG. 1), a system model, a processor and an output means.

FIG. 3 is a flow chart depicting the steps taken to achieve accurate measurement of an attribute or parameter with a system model created to represent the target according to the invention.

FIG. 4 represents aspects of a segmented scan and distribution of scatterers.

FIG. 5 depicts a model with 100 evenly spaced scatterers.

FIG. 6 depicts a model with 6 evenly spaced scatterers.

FIG. 7 illustrates a 10-micron offset of the model depicted in FIG. 6.

FIG. 8 represents generating segmented scan signals useful in creating a system model according to the invention.

FIG. 9 depicts an alternate embodiment of the system depicted in FIG. 2, wherein output further includes display of visual representation of the target, generated from the system model.

FIG. 10 depicts a depth scattering profile of a tissue target, according to one embodiment of the invention.

FIG. 11 depicts an eye as a target according to one embodiment of the invention, with examples of particular components including the lens, the cornea and the retina.

### DETAILED DESCRIPTION OF THE PREFERRED EMBODIMENT

Introductory remarks. Those of skill in the art can appreciate that the invention applies to a variety of devices that non-invasively obtain interferometric signals. Moreover, a variety of radiation interactions with a target of interest are likewise to be included in the inventive system and method. For convenience, and not to be construed as in any way limiting the scope of the invention, the detailed discussion provides examples pertinent to OCT, where the radiation is light.

Further, specific examples are provided with respect to a target, where the target is in vivo tissue, and where the attribute of interest is an analyte, specifically, glucose concentration. However, references to the target as “tissue” are for ease of comprehension, and the reader is reminded that “tissue” in the preferred embodiment does not in any way limit the target as contemplated according to the inventive system and method.

Owing to the fact that the most prominent interaction between light and human tissue is scattering the model of radiation interaction with the target may sometimes be referred to herein as “field of scatterers” [or sometime, a scattering model]. This reference, too, is illustrative and for convenience, and is not to be construed as a limitation on the target model.

Reader is reminded that the invention provides a heretofore unavailable means to generate a complete target profile or representation from incomplete information. Although the discussion describes the invention with respect to a segmented scan performed by an OCT, those of skill in the relevant arts will appreciate that partial target profiles obtained from a variety of analysis systems and method benefit from the invention taught herein.

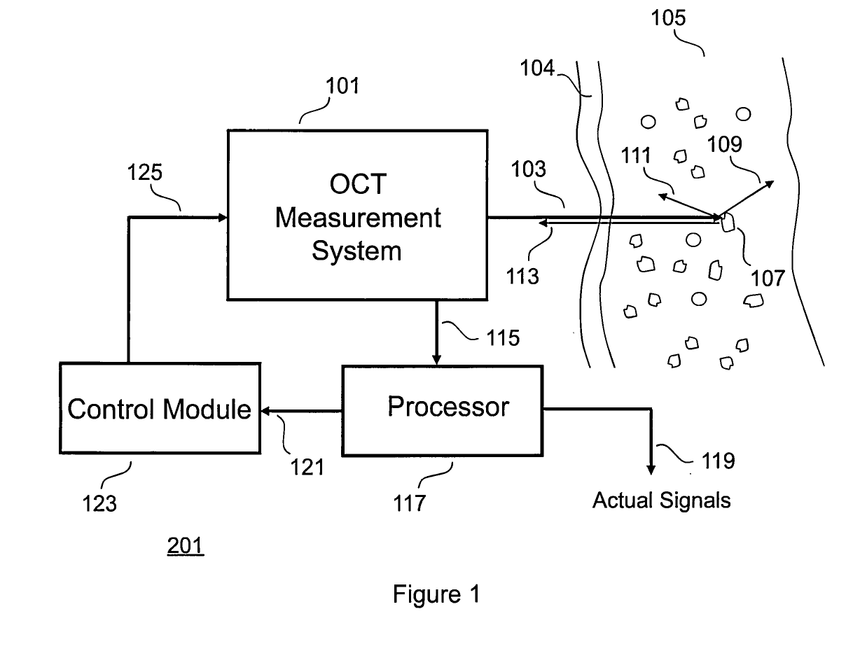

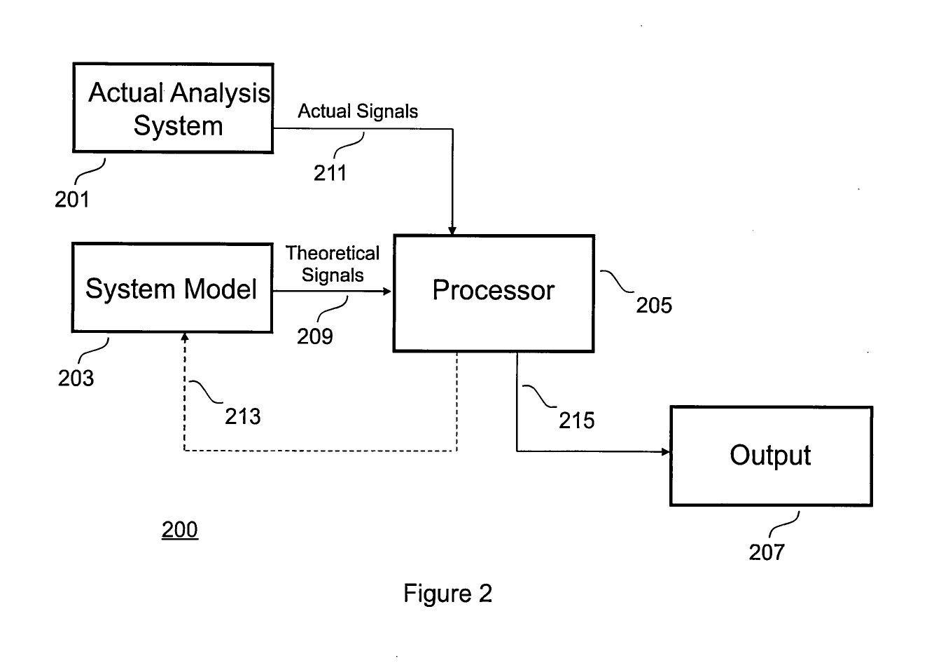

The preferred embodiment of the inventive analysis system is illustrated in and described with respect to FIGS. 1 and 2. In FIG. 1 an OCT measurement system 101 directs light 103 through the skin 104 into the tissue target 105. For purposes of this invention, tissue includes all components associated with human tissue including, but not limited to, cells, cell membranes, interstitial fluid and blood.

Light is scattered due to refractive index discontinuities at boundaries of tissue components (e.g. component 107). The scattered light can be in any direction, indicated by 109 and 111. Some light is back-scattered substantially along the direction 113 of the light directed at the tissue, to generate interference signals in the OCT measurement system 101. Such light can be back-scattered due to single or multiple scattering events, i.e. due to ballistic photons or multiple photon scattering.

The resulting optical interference signals are detected by one or more detectors to produce analog electrical signals 115. It can be appreciated that the output need not be analog i.e. the A-D conversion could be included in the detection process. Analog electric signals are typically digitized and under go some processing, also referred to as pre-processing, in a processing module 117. The resulting pre-processed digital signals are referred to herein as actual signals 119. Actual signals 119 contain information related to an analyte of interest (ex. glucose concentration). In alternate embodiments, actual signals may contain image-related information, permitting a visual representation of the tissue under examination, or may contain information relating to statistical distribution of scatterers in a target of interest.

The processor 117 may also provide feedback signals 121 to a control module 123 that controls the performance of the OCT measurement system 101 by means of control signals 125. Such control signals can include, but are not limited to, temperature control signals, one or more piezo drive signals and signals to control lateral scanning of the OCT measurement system 101. The combination of the OCT measurement system 101, the processor 117 and the control module 123 is referred to herein as the actual analysis system 201, depicted in FIG. 2.

A preferred embodiment of a non-invasive analysis system according to the invention is illustrated in and described with respect to FIG. 2. The analysis system is comprised of an actual analysis system 201, a system model 203, a processor 205 and an output means 207.

In the preferred embodiment, an actual analysis system 201, FIG. 1 creates interferometric signals. The interferometric signals are detected as analog signals and typically digitized and undergo pre-processing where such pre-processing may include filtering and the like. Such pre-processed, digitized signals are referred to herein as actual signals 211. Actual signals 211 output from the actual analysis system 201 are sent to the Processor 205.

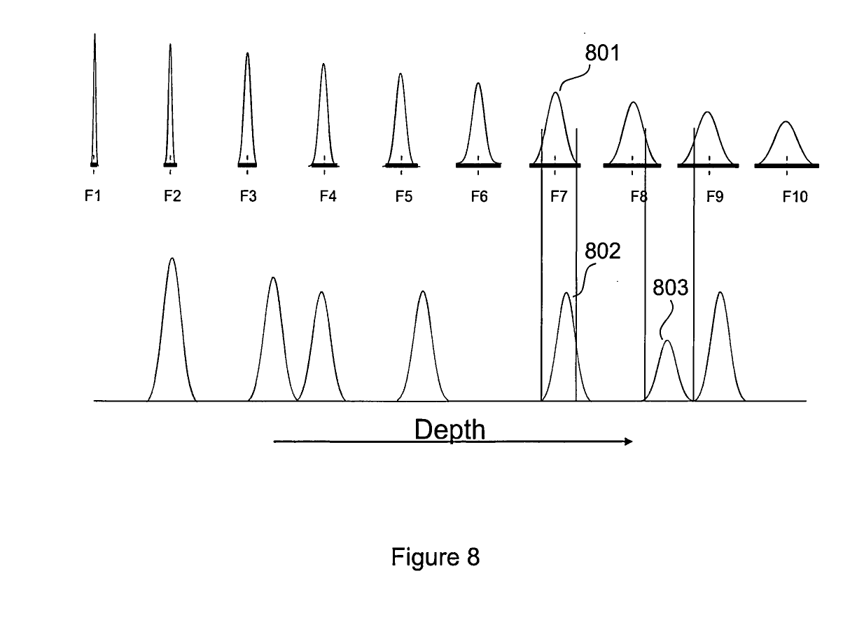

The system model 203 is comprised of a representation of tissue, the characteristics of the actual analysis system 201 (for example, center wavelength, bandwidth, power, speed and magnitude of piezo motion). In the invention, the characteristics are from the actual opto-mechanical system itself or a mathematical description of the parameters such as wavelength, etc. As depicted in FIG. 8, line 801 represents an ideal set of rectified interference signals that would be generated by ideal scatterers (i.e. of known location, intensity and phase) and interacts such interference signals with signals generated by actual scatterers in the target of interest (i.e. tissue components of unknown location, intensity and phase) represented by line 802. The system model can locate scatterers at any region within the target, including regions that are not actually scanned by the non-invasive analysis system and can include the influence such “un-scanned” scatterers would have on the theoretical interferometric signals it generates. By comparing such theoretical signals with actual signals a more complete representation of the target can be generated from the incomplete information of a non-continuous scan.

The system model 203 generates and outputs at least one theoretical signal 209, which is sent to the processor 205 that also receives actual signals 211. The theoretical signals 209 generated by the system model 203 are an ideal representation of the signals resulting from the interaction of radiation from an ideal analysis system with an ideal target: the ideal target is represented as a field of scatterers. As used herein, a field of scatterers will consist of scatterer location, scattering intensity and phase information and optionally absorption information. From the system model 203 theoretical signals can be calculated and sent to the processor 205.

Scatterers in between the segments of the scan will have some effect on the actual and theoretical signals due to such aspects as the low coherence length of the optical radiation or the reduction in optical intensity due to such scatterers, or multiple scattering events involving a scatterer in the gap. Fitting the theoretical signals to the actual signals extracts or generates probabilistic or most likely representation of the gap region.

The processor 205, which may be a micro-processor or DSP (digital signal processor), such as an ARM processor or a processor of the Blackfin family manufactured by Analog Devices, receives the actual signals 211, the theoretical signals 209. In the preferred embodiment, a model inversion approach is used—determining from the actual signal the field of scatterers that would result in such an interferometric pattern.

Alternatively, the processor 205 iteratively adjusts the parameters of the system model 203 so that the parameters of the field of scatterers and, consequently, the theoretical signals, 209 match the actual signals 211.

In another alternate embodiment, the system model 203 includes a noise model. Adjusting the parameters of the system model 203 to get a best fit between the actual signals 211 and the theoretical signals 209 and to best match the noise characteristics of the predicted or measured noise yields an optimal value of one or more system model 203 parameters. Adjusting the parameters of the system model 203 to get a best fit between the actual signals 211 and theoretical signals 209 and also to match the statistical characteristics of difference between the actual and theoretical signals noise characteristics of the predicted or measured noise yields an optimal value of one or more system model 203 parameters.

As has already been stated, adjustment of system model parameters may be an iterative process with repeated optimization of one or more parameters and feeding back one or more adjusted model parameters 213 to the system model 203. The system model may be dynamically selected from a set of pre-existing model templates (e.g. based on target type, regions of tissue or other characteristics of the target). The system model may be generated based on an understanding of the physics of the light interacting with the target. The system model may be empirically generated by analyzing data sets, such that a pattern is found dynamically without necessarily being predicated on the operative physics.

It can also be appreciated that various combinations of understanding of the operative physics along with iterative outputs of the processor using signals from multiple targets where multiple targets may include multiple target sites on the same individual and target sites on multiple individuals or any combination thereof.

Estimation techniques to optimize the fit of theoretical signals (and hence the field of scatterers representation) to actual signals. Estimation techniques include but are not limited to: maximum likelihood techniques; least mean square techniques; weighted least mean square techniques; Bayesian inference; minimum of margin.

In an alternate embodiment, wherein the system model includes a noise model, estimation techniques to optimize the fit to measured data and noise characteristics, include but are not limited to: maximum likelihood techniques; least mean square techniques; weighted least mean square techniques; Bayesian inference; minimum of margin.

At least one of the model parameters 213 which contains information about at least one attribute of the target of interest, is also sent to an output module 207. The attribute of interest 215, which in the preferred embodiment is a glucose concentration related parameter, may be stored, displayed or made available for other operations which include, but are not limited to: controlling a device such as an insulin pump; or causing a cell phone to send a text message or pre recorded message; or controlling operation of a consumer device, such as an iPOD.

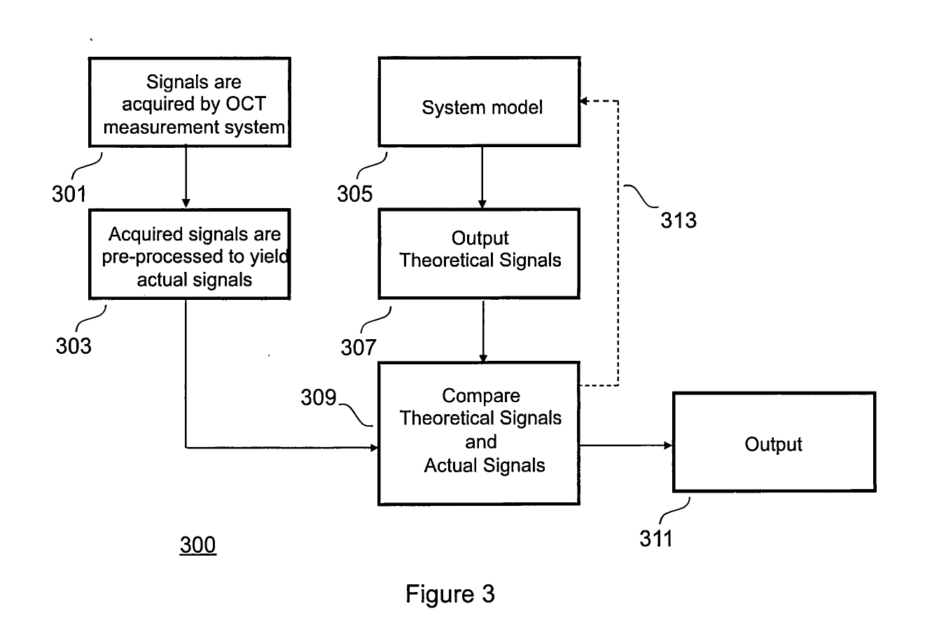

A preferred embodiment as to the inventive method of tissue analysis is further described with respect to the flow chart in FIG. 3 which depicts a preferred embodiment of the inventive method 300, comprising the steps set for the herein below. One or more interference signals are acquired by the OCT measurement system 301 as a result of being detected by one or more opto-electronic detectors. In the preferred embodiment the interference signals may be composite interference signals containing information related to multiple depths within the target of interest (as described in patents and applications incorporated herein by reference).

Detected interference signals, signals acquired by OCT measurement system 301, i.e. detected interference signals, are acquired signals. Such acquired signals, are pre-processed/processed to yield actual signals 303. Such pre-processing may include the sub-steps of: analog filtering the detected signals; digitizing the filtered detected signals; time domain digital filtering; frequency domain filtering including Fourier transform processing and periodogram processing; envelope detection; windowing to extract a desired portion of the filtered raw; various combinations of correlating and averaging spatially related signals; time-frequency processing, such as wavelet transforms. Note that windowing, for example, may be used to extract data during a linearized portion of a modulating signal (such as a Piezo drive signal). Pre-processing may also include linearization of the data to compensate for non-linearities of the modulated signal. In the an embodiment, the periodogram of the pre-processed raw data is computed, typically by calculating the square of the fast Fourier transform (FFT) modulus of each scan or of a set of combined scans to form processed raw data. The resulting periodogram may be normalized. Scans may be split into sub-scans to improve the periodogram SNR, if needed or/and several successive scans can be combined to improve the SNR.

Referring again to FIG. 3, the step of generating a system model 305 provides an ideal version of actual signals, i.e. processed signals produced by the actual OCT measurement system. The system model has already been discussed with respect to FIG. 2, 203. The output of the system model 305 is theoretical signals 307 which are idealized actual signals. Various ways of selecting or generating the system model are discussed above. This model can include parameters related to the OCT measurement system, such as, the variation of intensity of different order reference signals determined by the reflectivity of a partial mirror and polarization effects (as described in U.S. Pat. No. 7,526,329 titled “Multiple Reference Non-Invasive Analysis System” and patent application Ser. No. 12/214,600, “Orthogonal Reference OCT System with Enhanced SNR”, both incorporated herein).

The U.S. Pat. No. 7,526,329 patent and Ser. No. 12/214,600 patent application describe generating multiple reference signals by means of multiple reflections between a partial mirror and a mirror mounted on a piezo device. The relative magnitudes or intensities of these multiple reference signals are determined by factors where such factors include the reflectivity of the partial mirror, and may include polarization characteristics of the piezo and partial mirrors.

These multiple reference signals will generate multiple interference signals, which in the preferred embodiment are detected as a composite interference signal. When processed by periodogram or Fourier domain techniques the interference signals are manifest as peaks centered multiples of the frequency related to the first order interference signal generated by the basic scanning of the modulating Piezo device. This can be seen by referring to FIG. 8, wherein lines 801, 802 depict the magnitude of signal coming from scatterers at different depths, denominated F1 through F10, where F1 is the shallowest, and F10 is deepest in a target of interest.

Referring again to FIG. 3, the step of comparing theoretical signals and actual signals 309 is performed, and the results of the step of comparing transmitted to an output means 311. In some cases, feedback from the step of comparing theoretical and actual signals 313 is sent to the system model 305. By means of such feedback 313, adjustments to the system model may be made, as has been discussed. The method provides for outputting 311 the results of the processing step and the output is the value of at least one attribute, feature, or statistical distribution of interest. The results of the processing step include generating model parameters. At least one of the model parameters includes information about at least one attribute of the target of interest and is sent to an output module. The model parameter, which in the preferred embodiment is a glucose concentration related parameter, may be output in a variety of ways, i.e. stored, displayed or made available for other operations which include, but are not limited to: controlling a device such as an insulin pump; or causing a cell phone to send a text message or pre recorded message; or controlling operation of a consumer device, such as an iPOD or cell phone.

In some embodiments, the output provided is data pertaining to statistical distribution, rather than analyte characteristics. It can be appreciated that raw statistical data may be presented to User arranged in scoring or ranking protocols developed for any particular application.

As previously discussed, not all embodiments of the inventive method employ iterative approaches. In a preferred embodiment, the target representation is generated by the processor comparing the ideal signals to the theoretical signals, and providing the best matching target model, without iterating the system model. In the example discussed herein below, where the analyte of interest is glucose concentration in human tissue, the processor employs a model inversion algorithm.

A model inversion algorithm for determining glucose using the system is described herein. This method for determining glucose concentration is based on modeling the tissue as a scatterer or reflector field, and analyzing the properties of the field.

The interaction of radiation and the field of reflectors is described below. The radiation is the optical beam from the OCT system. Neglecting for the moment the optical beam width, if z denotes depth, zᵢ the depth of the i'th reflector, and aᵢ the energy reflected by the i'th reflector, the received time signal can be modeled as,

s(z)=(Σᵢaᵢδ(z−zᵢ))*g(z)+v(z) (1)

For convenience it is assumed that time is appropriately converted to depth due associated with the mirror scan mechanism so that the signal may be directly written in terms of depth. In Equation (1), δ(z) is the Dirac delta function, “*” is convolution, and g(z) the “speckle kernel” representing the optical system. The speckle kernel is Gaussian with zero mean and variance determined by the SLD bandwidth. The variance can be given either by knowledge of SLD properties, or estimated from a test scan using a mirror as target. Finally, v(z) represents a noise term due to various sources.

We further denote the reflector field by,

h_{f}(z)=Σᵢaᵢδ(z−zᵢ) (2)

so that (1) may be written as

s(z)=h_{f}(z)*g(z)+v(z) (3)

In this form, determining the reflector field is carried out by one of several standard deconvolution algorithms that exist in the literature. For example, as in [reference Blu, Bay and Unser, 2002].

To apply the model (3) in the inventive system described herein, we must take into account that multiple reflections are simultaneously received. Mathematically this is expressed as

s(z)=Σᵣ[h_{f}(sᵣz)*g(sᵣz)]wᵣ(z)+v(z) (4)

where the sum is taken over all reflections r considered to have non-negligible energy (i.e., above the noise floor), sᵣ is the scale factor due to the reflection r, and wᵣ is the rectangular window function taking into account gaps and overlap of the r'th reflection along the depth axis. The model inversion goal is now to estimate the reflector field (2) based on the actual received signal and the model of the actual received signal given by (4).

The novel model inversion as carried out in the inventive tissue analysis method adapts techniques used in, for example, fields such as Super-Resolution Video Reconstruction [reference Blu, Bay and Unser, 2002]. The model inversion method will involve discretizing (4) and representing equation (4) in matrix form.

After the model inversion has been done, it remains to use the reflector field to determine characteristics of analyte of interest (for example, in the case of glucose as the analyte of interest, to determine glucose concentration). There are multiple possible processing applications suitable for different circumstances, depending on the number of scatters present and the effect of analyte on the reflecting or scattering distribution.

For example:

Case 1. If the presence/concentration of an analyte changes i in (2) then such change may be tracked. If when glucose is present, there are a greater or lesser number of terms i in (2), attributable to the glucose then in this case glucose can be tracked by the number of terms required for the model to invert properly.

Case 2. If the concentration of glucose does not effect the number of terms i in (2) regardless of analyte content, it may be tracked by the exponential decay represented in the aᵢ terms.

Case 3. If there are relatively few terms reliably estimable, and these are due to tissue structure then the concentration of analyte of interest is determined by maintaining a rough map of these tissue structures, and noting the falloff in aᵢ terms. The assumption is that the ratio (or falloff) between structures is due to the impact of the concentration of the analyte of interest on transmission. This has been observed to be the case with respect to glucose concentration in human tissue.

Moreover, it can be appreciated that with different targets, and with various types of segmented scans, different approaches will empirically develop. Scan types that may be treated for the purpose of this invention as segmented, and which benefit from the inventive system and method include the flowing types: i. segmented depth scan; ii. stepped lateral scan; iii. in Fourier domain system: discrete wavelength steps, either with a stepped tunable source, or with a segmented detector array.

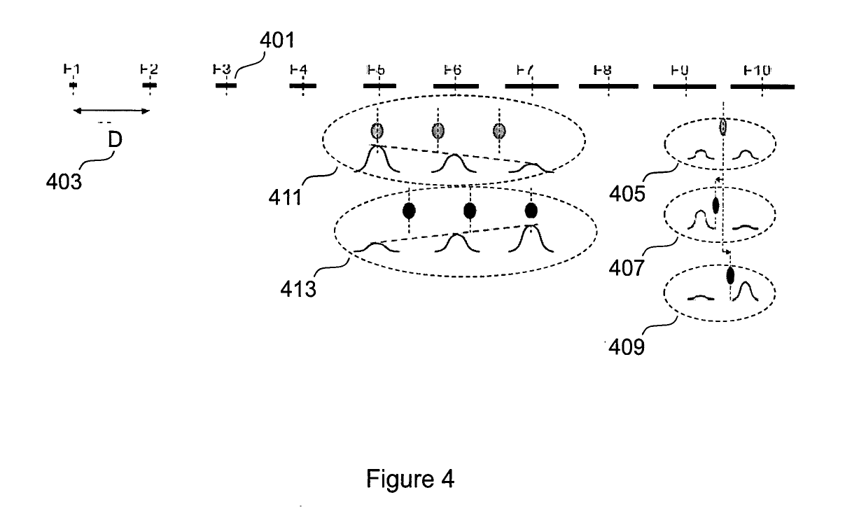

A discussion of FIG. 4 through 9 is presented herein as an aid to more fully appreciate aspects of the invention, and the problems solved by the invention.

FIG. 4 represents aspects of a segmented scan and distribution of scatterers. Areas labeled F1 through F10 represent sub scans. The scans are centered on distance D 403, where D is equal to separation distance between the midpoints of the scan segments, F1 and F2, and so on through F9 and F10. The subscans depicted by the darker horizontal lines, increase in magnitude such that F3 401 is three times longer than F1, so the gap decreases and eventually leads to overlapping scan segments.

Alignment of scatterers. In the case where a scatterer is at or near the midpoint of a gap between scan segments, as depicted by 405, the scatterer can have an effect on adjacent scans. In this case the resulting signal can contribute substantially equally to the scans to left and to the right of the scatterer. If however the scatterer is located closer to scan F9 then its contribution to or influence on scan F9 will be substantially greater that its influence on scan F10 as depicted by 407. Similarly if the scatterer is located closer to scan F10 then its contribution to or influence on scan F10 will be substantially greater that its influence on scan F9 as depicted by 409. Consequently, as can be appreciated by comparing 411, a negative slope owing to the position of the scatterers, with 413, a positive slope caused by the effects of a slight shift of the scatterers to the right. Such a slight shift could readily be caused by a slight change in the alignment of the non-invasive analysis system with the target.

This illustrates that alignment of scatterers is very important is segmented scans. Scatterers and individual alignment can affect slope. It can be appreciated that in addition to depth segmented scans, scanning laterally in discrete steps encounters the same difficulties. Thus the inventive system and method provide a valuable solution to extracting reliable information from segmented scans, whether depth scans or lateral scans.

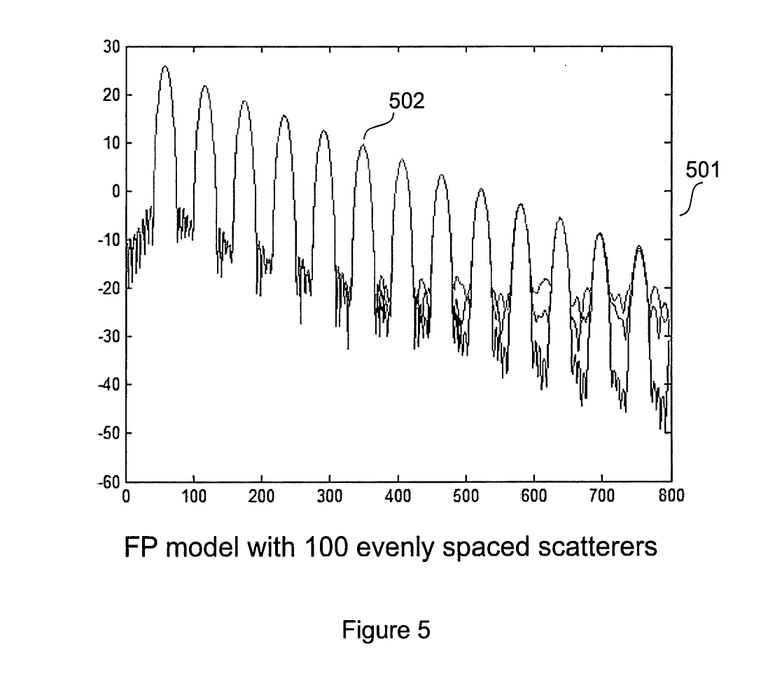

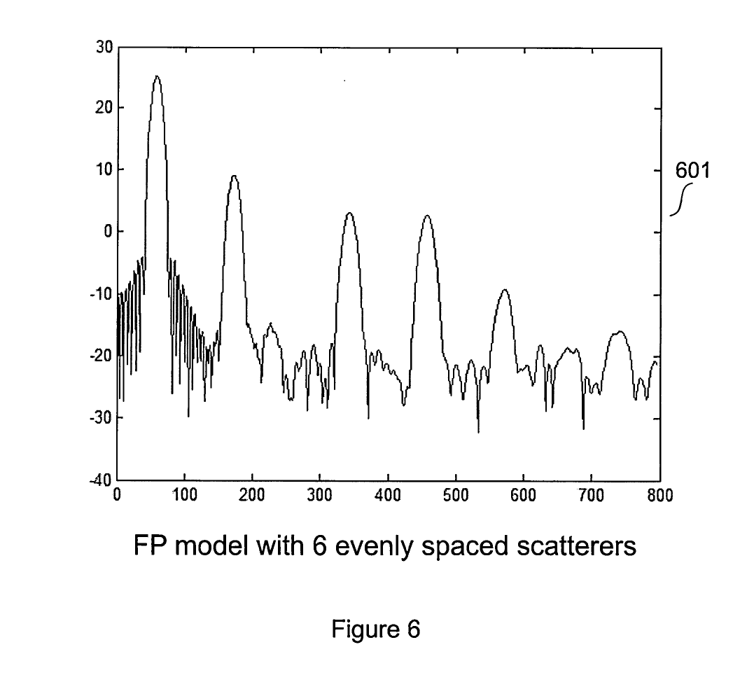

FIGS. 5, 6 and 7 further illustrate the signal sensitivity of scatterer distribution and alignment. 501 of FIG. 5 depicts theoretical signals generated by the system model with a field of scatterers consisting of 100 evenly spaced scatterers. For example 502 is one peak of a set of peaks with a relatively uniform negative slope. 601 of FIG. 6 depicts theoretical signals generated by the system model with a field of scatterers consisting of six evenly spaced scatterers.

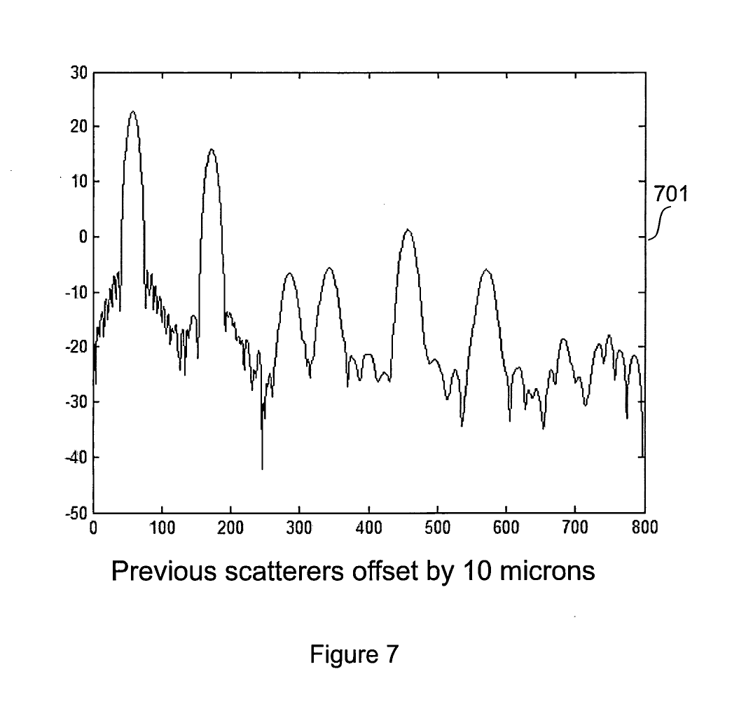

701 of FIG. 7 depicts theoretical signals generated by the system model with the same field of scatterers (consisting of six evenly spaced scatterers) offset from the field of FIG. 6 by 10 microns. Comparing FIG. 6 with FIG. 7 clearly illustrates the significant effect of scatterer alignment with the segmented scan. The difference between these scans provides information which can be used by the inventive processing solution to generate information related to the target characteristic in the gap. (i.e. complete representation from the incomplete information of a non-continuous scan). It can also be appreciated that alignment of scatterers with adjacent lateral scans or with the various segmented Fourier domain scans will similarly affect signals and be similarly amenable to the same inventive processing solution.

FIG. 8 further represents generating segmented scan signals useful in creating a model according to the invention. The signals F1, F2, F3, . . . F10 represent the multiple reference signals (one of which is 801) generated by the multiple reference OCT system. The lower set of peaks (one of which is 802) represent scattering signals from a random distribution of scatterers located in the target (deeper regions moving leftward). An actual interference signal would be related to the degree of overlap between, for example 801 and 802. It can be appreciated that as previously discussed with respect to FIG. 4 the peak 803 which is attributable to a scatterer in the “gap” between F8 and F9 will influence the interference signals associated with F8 and F9.

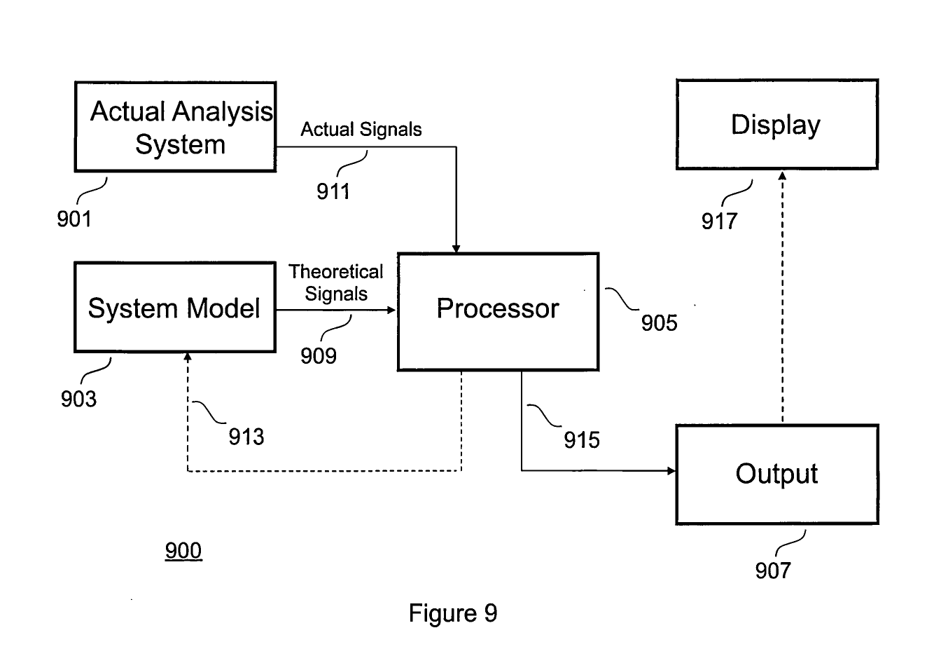

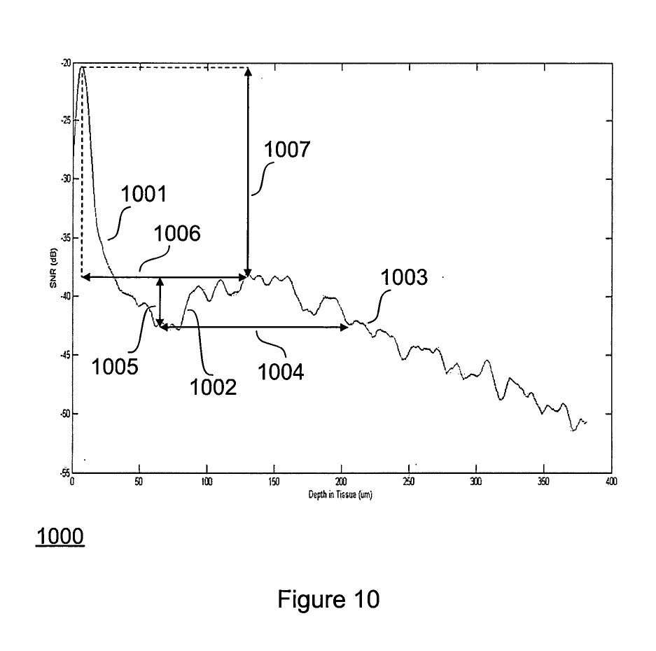

FIG. 9 depicts an alternate embodiment of the system 900 depicted in FIG. 2, wherein output 907 further includes a display means 917. In this embodiment, using the system model 903 to model a more complete representation of the target, the target can be imaged and the image displayed—i.e. readily output in a visual representation of the target, enabling visualization of, for example, a tomographic slice. It can be appreciated that as the data supports three dimensional imaging the capabilities of the display means could enable three dimensional or holographic images. More discussion regarding imaging output according to the invention appears in the discussion of FIG. 10. For elements of FIG. 9 not discussed, here, the discussion of corresponding elements in FIG. 2 applies, where, for example, the system model is number 203 in FIGS. 2, and 903 in FIG. 9, as are all elements appearing in both figures.

FIG. 10 depicts a scattering profile associated with human tissue composition at different tissue depths. A first, second, and third segment of a tissue scattering profiles 1001, 1002 and 1003 represent actual data from OCT on human tissue, at depth indicated on the horizontal axis. Information can be extracted from the segments of the scattering profile. The invention further provides a means by which information can be extracted from relative characteristic such as: the ratio of a width 1004 and a height 1005; or the ratio of a width 1006 and a height 1007; or other relationships that are known or are found to be meaningful. Such information may, for example, be related to analytes and the analyte may be glucose.

It is known that tissue, including human tissue, presents OCT scattering patterns consistent with actual tissue structure. See, for example, Alex et al. Multispectral in vivo three dimensional optical coherence tomography of human skin,” Journal of Biomedical Optics, 15(2) 026025 (March/April, 2010). Using a 1300 nm OCT system with a fiber laser-based source, the morphology of epidermis, dermis and sub-cutaneous layers could be visualized and delineated owing to pronounced differences in scattering. Differences in scattering attributable to a variety of factors including hairy skin, skin pigmentation, fatty skin, as well as skin location are observable.

One embodiment according to the invention includes in the target model, representation of tissue as a three dimensional field of scatterers, where one or more regions of the field of scatterers may be grouped or blocked as representing scattering patterns associated with tissue structures. By accounting for known tissue structures, the number of parameters in the target model may be reduced. To the degree that a target model may be composed of groupings relating to actual components within the target, the target model benefits from simplification, and improved accuracy. Moreover, in the preferred embodiment of the invention wherein an inverse model is employed to directly determine an attribute of interest, grouping representing of known tissue components is instrumental in providing a unique solution to the transform, as it aids in eliminating all but one solution from the set of possible solutions.

As illustrated in this discussion of FIG. 10, it can be appreciated that using three-dimensional field of scatterers as model for representing tissue, further permits exploiting general characteristics of tissue structures as additional constraints. Additional constraints increase accuracy by decreasing the number of possible variables.

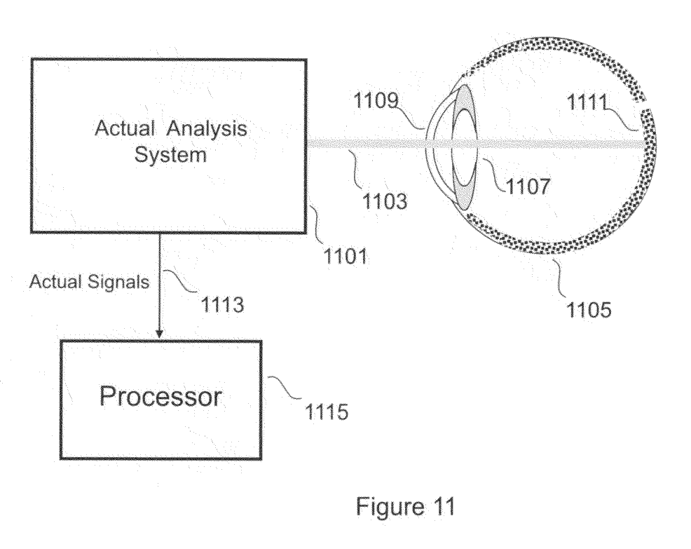

Another example, in the ophthalmic field, is illustrated in FIG. 11 where an actual analysis system 1101, such as an OCT measurement system, uses an optical beam 1103 to analyze an eye 1105. Components of the eye 1105, such as, for example, the lens 1107, or the cornea 1109, or the retina 1111, can be defined by a small number of parameters. For example in the case of the lens, the lens could be defined in terms of the curvature of both surfaces, its thickness and diameter. As described before, actual signals 1113 from the actual analysis system 1101 are sent to the processor 1115 to be processed.

In no way limited to the examples set forth herein, one must appreciate that the invention provides for accurate determination of target topology. Once an accurate topology has been generated, a variety of outputs are enabled by the invention. An analyte of interest (ex. analyte concentration in target tissue) can be determined. Alternatively, from the three-dimensional model of the target generated by the system model, an image of the scanned target made be displayed, with the User selecting any desired aspect of the display, from a tomographic slice, to a three dimensional projection, rotatable, and manipulable as any three dimensional holo-graphic image. Further, statistical distribution data may be output, from which a range of applications stem, including structure evolution for malign or cancerous elements.

It is understood that the above description is intended to be illustrative and not restrictive. Many variations and combinations of the above embodiments are possible. Many of the features have functional equivalents that are intended to be included in the invention as being taught and many other variations of the above embodiments are possible. Some further embodiments contemplated within the scope of the invention follow in the discussion hereinbelow.

The preferred embodiment above describes the invention in relation to a non-invasive analysis system, such as described in U.S. Pat. No. 7,526,329 titled “Multiple Reference Analysis System”, and further in U.S. patent application Ser. No. 12/584,666 and foreign counterpart PCT/US09/005,088, (“Noise Tolerant Measurement”) incorporated herein by reference. The invention is also applicable to conventional OCT systems that translate a single reference mirror or use other conventional technologies, such as fiber stretchers or rotating diffraction gratings to achieve depth scans of tissue.

The invention is applicable to many different types of non-invasive analysis systems based on OCT systems including, but not limited to conventional time domain scanning OCT; various multiple reference based systems; Fourier OCT using either a wavelength swept source or spectral OCT using a diffraction grating to separate wavelengths.

The embodiment described uses optical radiation, however the invention is not restricted to optical radiation. The invention could use other forms of radiation, including but not limited to, acoustic radiation such as ultra-sound, and other forms of electromagnetic radiation such as microwave or x-ray radiation. It could also use combinations of acoustic and optical radiation.

The invention is also applicable to non-invasive analysis systems for measuring glucose concentration, including but not limited to; reflective and transmissive spectroscopic approaches; photo-acoustic approaches; non-optical approaches, such as RF spectroscopy or other approaches based on measuring electrical properties of tissue or skin surface; thermal measurement approaches.

The invention is also applicable to invasive or minimally invasive analysis systems for measuring glucose concentration, including but not limited to; in-dwelling or implanted monitors; trans-dermal monitors that induce fluids through the skin surface to make glucose concentration measurements.

Furthermore, the invention is applicable to non-invasive analysis systems for measuring target properties that include concentration of analytes other than glucose. Moreover, the invention is not intended to be limited to use on human targets, but should include veterinary, agricultural and botanical applications. Other examples of application of the invention will be apparent to persons skilled in the art. The scope of this invention should be determined with reference to the specification, the drawings, and the appended claims, along with the full scope of equivalents as applied thereto.

For avoidance of doubt, it should be understood that the inventive applications as enabled by the invention set forth herein provides for accurate determination of target topology. Once an accurate topology has been generated, a variety of outputs are enabled by the invention. As illustrated by an example herein, an analyte of interest (ex. analyte concentration in target tissue) can be determined. Alternatively, from the three-dimensional model of some target of interest generated by the system model, an image of the scanned target made be displayed. With respect to such imaging, it should be appreciated that the User selects any desired aspect of the display, whether a tomographic slice or a three dimensional projection, such display rotatable, and manipulable as any three dimensional holo-graphic image. Further, statistical distribution data may be output, from which a range of applications stem, including structure evolution for malign or cancerous elements.

It is understood that the above description is intended to be illustrative and not restrictive. Many variations and combinations of the above embodiments are possible. Many of the features have functional equivalents that are intended to be included in the invention as being taught and many other variations of the above embodiments are possible.

1. A method performable by a non-invasive analysis system to determine at least one attribute of a target, said method comprising:
generating at least one actual signal from signals acquired by an actual system from said target, where said actual system is a non-invasive analysis system;
generating at least one theoretical signal by means of a system model that represents the interaction of radiation and said target, said system model comprised of:
said actual system characteristics;
a target model, said target model including at least one representation of said target, said representation describing said target as at least a one dimensional model of the interaction of radiation and said target,
processing said actual signal and said theoretical signal,
determining said attribute of said target, and
communicating said attribute to User.

2. The method as in claim 1 wherein the step of processing said actual signal and said theoretical signal includes generating a complete profile of target from information from a segmented scan of said target.

3. The method as in claim 1, wherein the step of processing said actual signal and said theoretical signal includes the sub-step of determining a target model with the target characteristics that match the actual signal.

4. The method as in claim 1, wherein the step of processing said actual signal and said theoretical signal includes the sub-step of providing feedback to said system model, where said feedback includes the comparison of said actual signal and said theoretical signal, and such providing feedback further enabling sub-steps of iterations of the system model, theoretical signal and processing steps, prior to the step of communicating said attribute of said target to User.

5. The method as in claim 1, wherein said target may include any of the following: tissue; tissue fluid; interstitial fluid; blood; eye; lens; cornea; retina.

6. The method as in claim 1 wherein said target model further includes grouping of radiant energy interaction characteristics, and where said groupings relate to actual components within said target.

7. The method of claim 1 wherein said attribute of said target may be any of: a target component of interest; an image of said target; statistical characteristics of said target.

8. The method of claim 7 wherein said target component of interest is an analyte of interest.

9. The method of claim 8 wherein said analyte of interest is glucose concentration.

10. The method as in claim 7 where at least one known property of said target is included in said target model, such that output communicating relative changes in interaction of radiant energy and said target may be associated with said attribute of said target.

11. The method as in claim 7 wherein said attribute of interest is an image of said target, and wherein said processing further including the sub step of generating a three dimensional model of the interaction of radiant energy with said target, said three dimensional model representing a complete description of said target, and wherefrom an image may be generated.

12. The method as in claim 11, further including the step of generating an image from said three-dimensional model of the interaction of radiant energy with said target, where the output is an adjustable display enabling selection of desired image and desired position of image.

13. The method as in claim 7 wherein said attribute to be determined is an obtained statistical distribution, such that said obtained statistical distribution is compared with a reference statistical distribution.

14. The method of claim 1, wherein said system model further includes a noise model.

15. The method as in claim 1, wherein the step of processing said actual signal and said theoretical signal includes estimation techniques to determine said attribute.

16. The method as in claim 15, where the attribute of interest is an analyte, and said analyte is glucose concentration.

17. A non-invasive analysis system comprising:
an actual analysis system, said actual analysis system outputting at least one actual signal, where said actual signal contains information obtained from a target of interest;
a processor, said processor including memory and capable of processing digital signals, and wherein said memory contains a system model, where said system model outputs at least one theoretical signal and where said system model includes:
said actual system characteristics;
a target model, said target model providing a representation of said target, said representation describing said target as at least a one dimensional model of the interaction of radiant energy and said target;
and where said processor compares said actual signal and said theoretical signal, and where said processor generates an output, where said output pertains to an attribute of interest of said target of interest.

18. The system as in claim 17 wherein said actual analysis system performs a segmented scan, and where said processor generates a complete profile of said target using information from said segmented scan.

19. The system of claim 17, wherein said processor selects from said system model a target model with the target characteristics that match the actual signal.

20. The system of claim 17, wherein said processor provides feedback to the system model of the comparison of said actual signal and said theoretical signal, enabling iterations of the system model, theoretical signal and actual signal processing.

21. The system of claim 17, wherein said target may include any of the following: tissue; tissue fluid; interstitial fluid; blood; eye; lens; cornea; retina.

22. The system of claim 17 wherein said target model further includes grouping of radiant energy interaction characteristics, and where said groupings relate to actual components within said target.

23. The system of claim 17 wherein said attribute of interest may include any of: a target component of interest; an image of said target; statistical characteristics of said target.

24. The system of claim 23 wherein said target component of interest is an analyte of interest.

25. The system of claim 24 wherein said analyte of interest is glucose concentration.

26. The system as in claim 23 where said target model includes at least one known property of said target, such that output communicating relative changes in interaction of radiant energy and said target may be associated with said attribute of said target.

27. The system as in claim 23 wherein said attribute of interest is an image of said target, said image generated from a three dimensional model of the interaction of radiant energy with said target, said three dimensional model representing a complete description of said target.

28. The system as in claim 24, wherein said image generated from said three dimensional model of the interaction of radiant energy with said target is output, said output providing an adjustable display enabling selection of desired image and desired position of image.

29. The system as in claim 28 wherein said attribute to be determined is an obtained statistical distribution, such that said obtained statistical distribution is compared with a reference statistical distribution.

30. The system as in claim 17, wherein said system model further includes a noise model.

31. The system as in claim 17, wherein said processor employs estimation techniques to determine said attribute.

32. The system as in claim 31, where the attribute of interest is an analyte, and said analyte is glucose concentration.
